(19) United States
(12) Patent Application Publication
(10) Pub. No.: US 2022/0359051 A1
(43) Pub. Date: Nov. 10, 2022

(54) Universal tinnitus management system (UTMS)

(71) Applicant: Aureliym GmbH, Bad Neuenahr-Ahrweiler (DE)

(72) Inventors: Christian Hauptmann, Bad Neuenahr-Ahrweiler (DE); Markus Haller, Bad Neuenahr-Ahrweiler (DE); Alexander Wegener, Bad Neuenahr-Ahrweiler (DE)

(21) Appl. No.: 17/635,788

(22) PCT Filed: May 14, 2020
(86) PCT No.: PCT/EP2020/063433

§ 371 Date: Feb. 16, 2022

(30) Foreign Application Priority Data
Aug. 16, 2019 (DE) 10 2019 005 768.6

(51) Int. Cl. G16H 15/00; G16H 80/00

(52) U.S. Cl. CPC G16H 15/00; G16H 80/00

(57) ABSTRACT

The present invention relates to an universal tinnitus management system (UTMS) for diagnosis and/or treatment of subjective Tinnitus, or for support of a health care professional (HCP) in the diagnosis and/or treatment of subjective tinnitus. The present invention further relates to the use of the UTMS for diagnosis and/or treatment of subjective tinnitus, or for supporting an HCP in the diagnosis and/or treatment of subjective tinnitus, a method of diagnosing and/or providing a therapy proposal for treating a subject having subjective tinnitus by means of the UTMS as well as a method of determining compliance with a therapy proposal and/or therapy progress by means of the UTMS.

The present invention resides in the field of tinnitus diagnosis and treatment and specifically concerns a universal tinnitus management system (UTMS) for diagnosis and/or treatment of subjective tinnitus, or for supporting a health care professional (HCP) in the diagnosis and/or treatment of subjective tinnitus.

Tinnitus affects about 10-15% of the population. Tinnitus involves the sensation of hearing sound when no external sound is present (phantom noise). The phantom noise may vary in pitch from a low roar to a high squeal, and it may be heard in one or both ears. In some cases, the sound can be so loud it can interfere with the ability to concentrate or hear external sound. Tinnitus may be present all the time, or it may come and go.

Subjective tinnitus is tinnitus only the affected patient can hear. This is the most common type of tinnitus. It can be caused by ear problems in your outer, middle or inner ear. It can also be caused by problems with the hearing (auditory) nerves or the part of your brain that interprets nerve signals as sound (auditory pathways).

A common cause of tinnitus is inner ear hair cell damage (e.g. caused by exposure to load noise or increased age). If the hairs inside the inner ear are bent or broken, they can pass random electrical impulses to the brain, causing tinnitus. Other causes of tinnitus include other ear problems, chronic health conditions, and injuries or conditions that affect the nerves in the ear or the hearing center in the brain. Less common causes of tinnitus include among others Meniere's disease and medications.

Tinnitus can significantly affect quality of life. Although it affects people differently, an affected patient may also experience fatigue, stress, sleep problems, trouble concentrating, memory problems, depression, anxiety and irritability. Treating these linked conditions may not affect tinnitus directly, but it can help the patient to feel better.

There is no single mechanism likely to explain the presence of tinnitus. As such there is no standardized management pathway of tinnitus. Choice of clinical intervention is therefore a multi-factorial decision based on many factors, including assessment of each individual patient needs and the respective healthcare context. This complexity may lead to the situation that the proposed therapy, and hence the efficacy of treatment, is highly dependent on the experience of the responsible health care professional and/or ear, nose and throat doctor. Moreover, patients that experience to little alleviation of their symptoms may try another professional in a hope to receive a more efficient treatment. This considerably increases the costs for managing tinnitus. Other patients may skip therapy sessions or give up completely because the sessions do not fit in their daily schedule.

The problem to be solved by the present invention is to simplify the complex management of subjective tinnitus. In particular, it was an aim to provide a more standardized approach in the assessment (diagnosis) and treatment of subjective tinnitus, to alleviate financial burden that the management of tinnitus has on the patient and/or healthcare system and to increase patient compliance with the proposed/prescribed therapy.

This problem is solved by an UTMS as disclosed herein, in particular as defined in the appended claims. The UTMS comprises:
a) an HCP component,
b) a plurality of patient components, and
c) optionally an ear, nose and throat doctor (ENT) component.

The UTMS is configured to facilitate communication between the HCP component, the plurality of patient components and, if present, the ENT component, via a server. Among this communication network, information can be reversibly transmitted (as explained further below).

The HCP component is configured to facilitate management of the plurality of patient components. The HCP component may provide an interface for an HCP in an application that runs on a terminal. The terminal may be a personal computer, a laptop, a notebook, a tablet or a mobile device. The application is preferably an app downloadable from an appstore but may also be an accordingly programmed website or a progressive web app. The HCP component may be configured to authenticate the HCP to the UTMS. For instance, the HCP component may be configured to request login credentials and transmit the entered login credentials to the UTMS (e.g. the server) for authentication. The UTMS preferably includes more than one HCP components, wherein preferably each HCP component is configured to authenticate the respective HCP to the UTMS.

An HCP as understood herein is a professional that is registered, approved or otherwise qualified to treat a patient with subjective tinnitus, such as a medical practitioner or an audiology professional or a trained acoustician.

The HCP component may include a dashboard as described further below. Moreover, the HCP component may implement at least one (for instance 2, 3, 4, 5, 6, 7, 8, 9, 10 or all) of the following functionalities: (i) accepting new patients, (ii) creating and managing accepted patients, (iii) accessing patients-specific data, (iv) displaying a plurality of therapy and/or diagnostic modules to be performed by a respective patient, preferably displaying the current, future and/or past therapy and/or diagnostic modules performed and/or to be performed by the respective patient, (v) displaying unanswered patient messages, (vi) arranging appointments between patients and HCPs and/or ENTs, (vii) displaying patient compliance and therapy progress, (viii) displaying background information and/or training material on diagnostic and therapeutic tools, (ix) displaying and/or defining inclusion & exclusion criteria for therapy proposals, and (x) assigning therapies to the respective patient.

Each of the plurality of patient components is configured to be assigned to a specific patient and to authenticate the respective patient component to the UTMS. Each patient component may provide an interface for a specific patient in an application that runs on a terminal. The terminal may be a personal computer, a laptop, a notebook, a tablet or a mobile device (preferred). A patient as understood herein means a subject that is suspected of having or has been diagnosed with subjective tinnitus. For authentication of the patient component to the UTMS, the patient component may be configured to request login credentials and transmit the entered login credentials to the server for authentication. The patient components are further configured to request patient-specific data from the respective patients (as further explained below).

The UMTS is configured to process the patient-specific data and communicate them to the HCP component and, if present and/or needed, the ENT component. To this end, the patient component may be configured to transmit the patient-specific data entered by the respective patient to the server for storage, processing and/or further transmission. Using the patient-specific data as an input to a predetermined therapy matrix, the UTMS is further configured to draw up a diagnosis and/or a therapy proposal.

The diagnosis and/or a therapy proposal determined by the UTMS may generally be used for diagnosis and/or treatment of subjective tinnitus directly. It is however preferred that the results are treated as preliminary and used to support a health care professional (HCP) in the final diagnosis and/or treatment of subjective tinnitus. For this purpose, the (preliminary) diagnosis and/or therapy proposal determined by the UTMS is transmitted to the HCP component for review by the HCP. Thereafter, the reviewed (and optionally adapted) diagnosis and/or therapy proposal is transmitted from the HCP component to the patient component. Whether the diagnosis and/or a therapy proposal determined by the UTMS is used as preliminary or final, the patient component is configured to display the (final) diagnosis and/or the (final) therapy proposal.

As an advantage, the UTMS is an easy to use, medical tool that allows a patient suffering from subjective tinnitus to be diagnosed and treated anywhere and anytime. Hence, the use of the UTMS is assumed to fit everyone's schedule. The UTMS combines advanced tinnitus assessment and established sound and psychological therapy. Moreover, by relying on a predetermined therapy matrix, the results obtained from the UTMS are highly reproducible and not based on subjective decisions. The UTMS may be implemented as a cloud-based system. The patient component may be implemented as a mobile phone on which an app is running that can be conveniently used at the patient's home and even on the patient's travels. Moreover, the patient can be supported by the HCP via the HCP component (e.g. implemented as a web app in a hearing care store) and, if necessary, by the ENT via the ENT component.

The ENT component, if present, may provide an interface for an ENT in an application that runs on a terminal. The terminal may be a personal computer, a laptop, a notebook, a tablet or a mobile device. The ENT component may be configured to authenticate the ENT to the UTMS. For instance, the ENT component may be configured to request login credentials and transmit the entered login credentials to the UTMS (e.g. the server) for authentication. The UTMS preferably includes more than one ENT component, wherein preferably each ENT component is configured to authenticate the respective ENT to the UTMS. The ENT component may further be configured to allow the ENT to access and display user-specific information. The ENT component may be further configured to receive and transmit data (such as results from an ENT assessment input by the ENT into the ENT component) to other components of the network such as the HCP component, the respective patient component and/or the server.

An ENT as understood herein is an ear, nose and throat doctor or similarly qualified professional.

For authentication, the patient component, the HCP component and/or the ENT component may use at least one authentication method provided by an operating system of a terminal on which the respective application is installed.

The UTMS may further comprise a system administrator component configured to administer the UTMS and keep the same operating. It may further comprise a data analyst component configured to evaluate the collected data for improvement of the UTMS.

As mentioned above, in one embodiment, the UTMS is configured to (directly) transmit the diagnosis and/or the therapy proposal over the communication network to the patient component of the respective patient. In this case, the diagnosis and/or the therapy proposal determined by the therapy matrix is displayed on the patient component without review of the results. The advantage of this embodiment is that patients not requiring an HCP can be diagnosed and/or treated without the help of an HCP. In a preferred embodiment, however, the UTMS is configured to communicate the diagnosis and/or the therapy proposal to the HCP component and/or, if present and needed, the ENT component for confirmation, rejection or revision of the diagnosis and/or the therapy proposal by a HCP and/or an ENT before the diagnosis and/or the therapy proposal is displayed on the patient component. In this case, the diagnosis and/or the therapy proposal determined by the therapy matrix is reviewed by an expert and the confirmed or revised diagnosis and/or therapy proposal is displayed on the patient component. Thereby, the advantages of the UTMS system can be combined with the advantages associated with an expert review of diagnostic results and proposed therapy. The confidence of the diagnosis and/or therapy proposal can thereby be further increased.

It is also envisaged that the UTMS determines based on the therapy matrix whether or not the diagnosis and/or the therapy proposal determined by the therapy matrix needs to be reviewed by the HCP and/or ENT. The advantage of such differentiation is that only specific, e.g., not clear-cut, cases are transferred to the HCP and/or ENT component. More patients can be managed by the same number of HCP components and/or ENT components.

The patient component may be configured to guide the patient through one or more diagnostic modules where the UMTS may record data via patient data entry (patient information, informed consent/HIPAA release, tinnitus questionnaires, etc.) and may have the patient perform certain assessments (i.e. diagnostic modules such as sound finder module, self-pitch-matching module, etc.). Hence, according to a further preferred embodiment of the present invention, the UTMS comprises at least one diagnostic module. The at least one diagnostic module serve the purpose of determining, conducting and/or receiving results from of at least one, preferably at least two, more preferably at least three, even more preferably at least four and most preferably all, of medical history, Tinnitus Handicap Inventory (THI), hearing assessment, tonality sound finder, medication use questionnaire, Tinnitus Functional Index (TFI), self-pitch matching, hyperacusis questionnaire, Hospital Anxiety and Depression Scale (HADS), Perceived Stress Questionnaire (PSQ), ENT assessment, hearing questionnaire, pitch quality, and minimum masking levels. The at least one diagnostic module may be implemented in the patient component in the form of a questionnaire, an analysis tool or the like. The patient component may then be configured to request the respective patient to complete the respective module, e.g. fill in the questionnaire and/or complete the analysis tool or the like. The data and/or results gathered this way from the respective patient's feedback to the at least one diagnostic module is to be understood as reflecting patient-specific data. The advantage of such guidance is an ordered workflow that ensures that the relevant diagnostic measures are taken at the right time and in the right order. This also helps to minimize the number of superfluous diagnostic measures, which might be ordered by some professionals on the basis of a merely subjective therapy selection.

The medical history module includes the patient's data regarding his case history, for instance, if he wears a hearing aid, which medication does/did he take etc. It can be implemented as a questionnaire displayed on the patient component.

The THI module is a questionnaire, which purpose serves the identification of difficulties that a patient may be experiencing because of tinnitus. The result is a score (THI score) ranging from 0 to 100. It can be implemented as a questionnaire displayed on the patient component.

The hearing assessment module is an audiological exam, whereby the patient's hearing is tested for irregularities or impairment. For instance, the hearing threshold for each individual ear and different sounds may be compared with results considered typical for the patient's age. This can help rule out or identify possible causes of tinnitus. As a result, the patient may be found to be normal hearing or to have irregular or impaired hearing. In the latter case, the patient component is preferably configured to display an invitation to visit the HCP for further testing. The hearing assessment can be implemented on the patient component, which may for this purpose be configured to broadcast audio signals to a headset of the patient. Accordingly, it is preferred that the patient component is functionally connectable to a headset.

The tonality sound finder module (also referred to as Tinnitus sound finder module) determines the nature of the tinnitus and identifies if it is a tonal tinnitus (including whistle, hum, or screech) or an atonal tinnitus (including white noise, crickets, or static). As a result, the patient may be found to have a tinnitus more of tonal nature or tinnitus more of atonal nature. The tonality sound finder may be implemented in the same way as the hearing assessment.

The medication use questionnaire module is a questionnaire, where the patient is asked to enter his medications including names, doses, regularity, etc. It can be implemented as a questionnaire displayed on the patient component.

The TFI module is a questionnaire having documented validity both for scaling the severity and negative impact of tinnitus for use in intake assessment and for measuring treatment-related changes in tinnitus (responsiveness). It provides comprehensive coverage of multiple tinnitus severity domains. The questionnaire results is a score (overall TFI score), whereby the sum of all valid answers from both TFI pages (maximum possible score=250 if the respondent were to rate all 25 TFI items at the maximum value of 10) is divided by the number of questions for which that respondent provided valid answers (yields the respondent's mean item score for all items having valid answers) and multiplied by 10. This results in an overall TFI score within a range of 0 to 100. It can be implemented as a questionnaire displayed on the patient component.

The (self-)pitch matching module is a diagnostic tool, whereby the patient matches the pitch of his tinnitus sound via the patient component. For instance, the patient component may be configured to continuously play a tone during the test and adapt the tone's frequency and intensity instantly in response to the patient's feedback. This functionality may be easily and user-friendly implemented by providing the patient component with a touch display on which the patient varies the tone by gestures until the patient thinks that he has found the closest match to the patient's tinnitus sound. The pitch matching modules comes with a functionality to assess the quality of the pitch match based on the recorded behavior of the patient during pitch matching. As will be understood, the functionality is not bound to a touch display but can also be implemented with any conventional display and input devices including a mouse or a keyboard. Audio signals may be emitted from the patient component but are preferably broadcast to the patient's headset.

The hyperacusis questionnaire module is a tool for measuring hypersensitivity to sound. It can be implemented as a questionnaire displayed on the patient component.

The HADS module is a measure of anxiety and depression in a patient. It can be implemented as a questionnaire displayed on the patient component.

The PSQ module is an instrument for assessing the stressful life events and circumstances that tend to trigger or exacerbate disease symptoms. It can be implemented as a questionnaire displayed on the patient component.

The ENT assessment module is an assessment that is carried out by an ENT or equivalent professional, for instance upon invitation displayed on the patient component. For this purpose, the patient component may be configured to invite the respective patient to visit an ENT, in order to examine the patient. Conveniently, the patient component also comprises an ENT finder module, which is configured to assist the patient in locating a nearby ENT. As described above, the ENT component may be configured to allow the ENT to access and display user-specific information, and to receive and transmit results from the ENT assessment to other components of the UTMS. Alternatively, the ENT can fill out a form, which is provided to the HCP who then inserts the information of the form into the UTMS, e.g. electronically by scanning the form.

The hearing questionnaire module is a measure of potential hearing problems. It can be implemented as a questionnaire displayed on the patient component.

The minimum masking levels module is a measure of the capabilities of broadband noise signals to partially or fully mask the tinnitus sensation. For instance, the patient component may be configured to continuously play a broad band noise signal to test and adapt the intensity instantly in response to the patient's feedback. This functionality may be easily and user-friendly implemented by providing the patient component with a touch display on which the patient varies the tone by gestures until the patient thinks that he has found the closest match to the patient's tinnitus sound. The minimum masking level module may come with a short questionnaire allowing the patient to indicate, if masking was observed. Additionally, the minimum masking modules may come with a functionality to assess the quality of the pitch match based on the recorded behavior of the patient during masking. As will be understood, the functionality is not bound to a touch display but can also be implemented with any conventional display and input device(s) including a mouse or a keyboard. Audio signals may be emitted from the patient component but are preferably broadcast to the patient's headset.

In order to prescribe the most suitable therapy to the patient, the patient may be invited to complete a certain number of (self-)diagnostic modules in a first step (herein also referred to as level I diagnostics). The level I diagnostics include at least one, preferably at least two, more preferably at least three and most preferably all of the THI, the hearing assessment, the hearing questionnaire, and the tonality sound finder modules. Preferably, not more than one diagnostic module can be completed in one day. The results of each diagnostic module are evaluated by the server and certain actions may be taken automatically, while some are taken after the HCP has reviewed them (as described herein). If needed, the patient component may invite the patient to repeat a diagnostic module.

Depending on the diagnostic results of the level I diagnostics evaluated by the server, the UMTS is configured to invite the patient to complete further diagnostic modules: If in the first step the respective patient is determined to have hyperacousis, and/or a THI index equal or above a predefined threshold (preferably equal or above 58) or a tonal tinnitus, the UTMS is configured to invite the patient to complete the hyperacousis questionnaire, the HADS and/or the PSQ questionnaire and/or the minimum masking levels and/or the pitch matching modules in a second step. If, on the other hand, in the first step the respective patient is determined to have no hyperacousis, and/or a THI index below a predefined threshold (preferably below 58), and no tonal tinnitus, the patient is invited to complete the minimum masking levels, the TFI and the medication use questionnaire modules in a third step. Optionally, the third step involves an invitation to complete the ENT assessment module. The diagnostics following the level I diagnostics are also referred to herein as level II diagnostics.

After the results of the level II diagnostics are evaluated by the server, the UTMS may propose a diagnosis and/or a therapy (also referred to as level III therapy). This means, the UTMS is configured to draw up the diagnosis and/or the therapy proposal in the fourth step. As mentioned above, the diagnosis and/or therapy proposal may be used as final or as preliminary, whereas in the latter case the HCP is able to finally approve, adapt or reject the diagnosis and/or therapy proposal. If the patient is determined by the server (and optionally approved by the HCP) to be suitable for treatment by the UTMS, the patient may receive an ENT form for completion by an ENT. If an ENT that might be associated with the HCP of the respective patient has been or is selected from the ENT finder module, the ENT may be granted access to the respective patient's data via the ENT component. He may then complete the ENT online and/or schedule an appointment with the respective patient. In another embodiment, the patient may print out the ENT form and gets it completed and signed by an ENT of choice of the patient, which thereby prescribes the UTMS therapy. The signed form can be photographed by the patient component and sent to the HCP component (and thereby to the HCP) or alternatively scanned and sent via E-mail or by land mail to the HCP.

In a particular embodiment of the present invention, the UTMS is configured to determine whether a therapy is contraindicated based on the predetermined therapy matrix and/or to determine weighting factors for different therapy options for assisting in therapy priorization based on the predetermined therapy matrix, and communicate the respective results to the HCP component. This embodiment may help to prevent an HCP from overruling the therapy proposal determined by the UTMS and select a therapy that, for the present patient, is contraindicated. Moreover, the most efficient therapy can be chosen first.

In a further embodiment, the UTMS comprises at least one therapy module and preferably two, three, four, five or six therapy modules. Each therapy module may serve the purpose of providing a part of the overall therapy proposal. The therapy modules may be selected from the group consisting of referral to an HCP for a hearing aid evaluation, patient education & counselling, cognitive behavior therapy (CBT), tonal sound therapy, masking (MML) and noiser usage module.

In the patient education & counselling module properly validated, up-date and scientifically founded information are provided in a patient friendly fashion. Patient information and support topics are preferably tailored to the patient's need and what is available to them. Possible topics include one or more of: (i) What is tinnitus? What causes and maintains it? (ii) Pulsatile tinnitus (follows heartbeat); (iii) Common misunderstandings and myths; (iv) Hearing loss and hearing aids; (v) Ear wax removal; (vi) Hyperacusis and tinnitus; (vii) Protecting your hearing; (viii) Habituating to tinnitus; (ix) Relaxation; (x) Monitoring tinnitus; (xi) Use of sound; (xii) Dealing with sleep problems; (xiii) Dealing with emotional consequences of tinnitus. The patient education & counseling module may consist of text information with diagrams and potentially several short sound or video sequences. Technically, education & counseling is similar to CBT.

The CBT module is a psycho-social intervention that aims to improve mental health. CBT focuses on challenging and changing unhelpful cognitive distortions (e.g. thoughts, beliefs, and attitudes) and behaviors, improving emotional regulation, and the development of personal coping strategies that target solving current problems. This module is very content intense and may feature a variety of images, text, sounds, videos, questionnaires and exercises to be done by the patient. Typical 1 h lesson may be executed in, e.g., 15 min “slices”. Remainders are provided to the patient to keep up with the program. Focus shall be on healthy mix of audio-visual and text in order keeps the patient's attention.

The masker module is a very powerful tool, in the first place to distract the patient's attention from the tinnitus sound and in some cases to induce temporal residual inhibition. During residual inhibition neuronal elements in the hearing system are activated, which cause a reduction of tinnitus intensity for several seconds to minutes after the presentation of an intense and short (e.g. 30 seconds) masking sound. The masker may be constructed using 5 noise bands. For each noise band the minimum masking level may be adjusted by the patient (for left and right ear separately). During presentation of all, e.g. 5, noise bands in the majority of patient's tinnitus is “masked” and difficult to hear for one of these noise bands. This might be an indicator of the location of tinnitus with respect to tinnitus. Thereafter, a signal may be constructed using the bands 2-5. When such a masker signal (bands 2-5 only) is presented with higher intensity for 30 seconds, in a larger portion (i.e. approx. 60%) of tinnitus patients tinnitus is absent after the masker signal stopped, this effect is called residual inhibition. Such tinnitus inhibition is not lasting and tinnitus reappears after several seconds or minutes. Thereafter, the full signal (5-bands for each ear) may be constructed and balanced. This signal may be used by the patient for several hours per day. Intensity of signal may be adjusted using the master volume.

With respect to the noiser module, it is noted that, if the patient suffers from Hyperacusis (as identified by the corresponding questionnaire), a noise may be the appropriate and clinically tested therapy. For this, a pink noise (1/f spectrum) may be used. The intensity of the noise may be adapted with the corresponding module. This signal may be used by the patient for several hours per day. Intensity of signal may be adjusted using the master volume.

The tonal therapy module may receive information about patient's tinnitus pitch from the pitch matching module, in particular the obtained thresholds and the tinnitus frequencies and intensities. The following procedures may be implemented and completed before therapy can be started: (1) Selection of ear to be stimulated (right, left, both); (2) Therapy signal calculation: a) Four therapy tone frequencies calculated for each ear; b) Selection of fixed therapy tone frequencies (e.g. 0.766, 0.9, 1.1, 1.4; other fixed tone frequencies are also possible) or hearing threshold adapted therapy tone frequencies; (3) Selection of therapy tone intensities (tone balancing), for each ear and between both ears (if both ears are stimulated). The tonal therapy module may have a graphical user Interface, which allows an intuitive use by the patient, use a clear design and guide the user through the workflow. The module may generate sound of high quality (e.g. high SNR>80 dB), have a high accuracy with respect to frequency (e.g. error <0.1%) and amplitude (e.g. error <1%). The stimulation signals may be windowed by a Hanning window to have a smooth on- and off-set of tones. The patient may be instructed by the patient component to adjust the tone intensities such that all four tones have approximately equal loudness for the patient. Once one ear is adjusted, the inter-ear balancing is done, e.g. by presenting two tones, one from the already adjusted ear and a second tone from the other ear. During this only one slider may be presented, allowing to adjust the new tone from the second ear. The patient may be instructed to adjust the tone of the other ear, such that both tones have approx. equal intensities. Once this inter-ear balancing is tone, for the second ear the tone balancing may be done. The balancing may be similar to the balancing of the first ear, but the one tone used for the inter-ear balancing may be fixed and cannot be changed, e.g. by only presenting three sliders.

If the hearing aid module is selected and hearing impairment was observed, the patient component may display a message suggesting that the patient visits the HCP for getting a hearing aid fitting.

In a case where the therapy proposal includes more than one therapy module to be performed by the respective patient, the patient component is preferably configured to provide an overview of the therapy modules to be performed.

To track patient compliance and/or therapy progress, the patient component may be configured to request patient-specific data at different time points and/or to track usage time if allowed by the patient in the corresponding consent form.

Another functionality of the UTMS concerns a payment module for billing the patient. To this end, the patient component is configured to request payment details from the respective patient.

In a preferred embodiment, the UTMS comprises a plurality of HCP components and the HCP component configured to facilitate management of the respective patient component can be chosen from the respective patient component using a HCP finder module. Moreover, each HCP component may manage a plurality of patient components.

In a preferred embodiment, the UTMS comprises a plurality of ENT components and the ENT component assigned to the respective patient component can be chosen from the respective patient component using an ENT finder module. Moreover, each ENT component may manage a plurality of patient components.

Another aspect of the present invention relates to the use of an UMTS as described herein for diagnosis and/or treatment of subjective tinnitus, or for supporting an HCP in the diagnosis and/or treatment of subjective tinnitus, as described herein.

Another aspect of the present invention concerns a method of diagnosing and/or providing a therapy proposal for treating a subject having subjective tinnitus by means of an UTMS as defined herein. The method comprises the steps: (i) registration of the subject as a new patient of the UTMS, (ii) providing patient-specific data (as disclosed herein) of the subject to the UTMS, and (iii) drawing up a diagnosis and/or a therapy proposal based on a predetermined therapy matrix using the patient-specific data as an input (as disclosed herein). The step of providing the patient-specific data shall be understood to be the result of the data collected by the UTMS during the guidance of the patient via the patient component through the diagnostic module(s) and optionally the therapeutic module(s) as described in the context of the UTMS of the invention.

A further aspect of the present invention relates to a method of determining compliance with a therapy proposal and/or therapy progress by means of a UTMS as defined herein. The method comprises the steps: (i) drawing up a therapy proposal for treating a subject having subjective Tinnitus by a method as defined above, (ii) tracking over time the subject's compliance with the therapy proposal and/or therapy progress using the patient component, and (iii) showing the compliance on the HCP component.

The various features defining the UTMS and the various embodiments of the UTMS shall be understood to define according features and embodiments of the use and the methods of the invention.

Further aspects, embodiments and advantages of the present invention become apparent from the following detailed description, the figures and claims, which follow after the brief description of the drawings.

In the drawings:

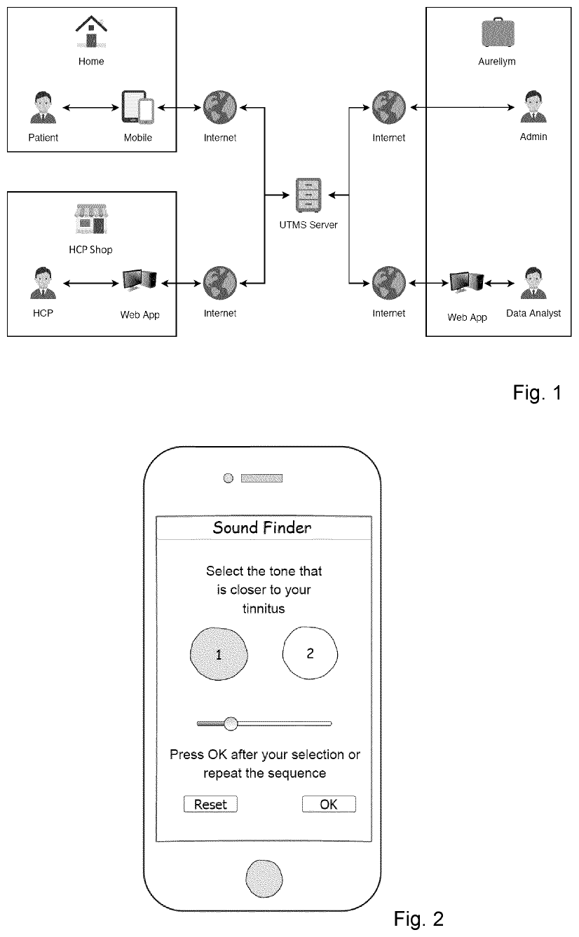

FIG. 1 shows an exemplary communication network of the UTMS.

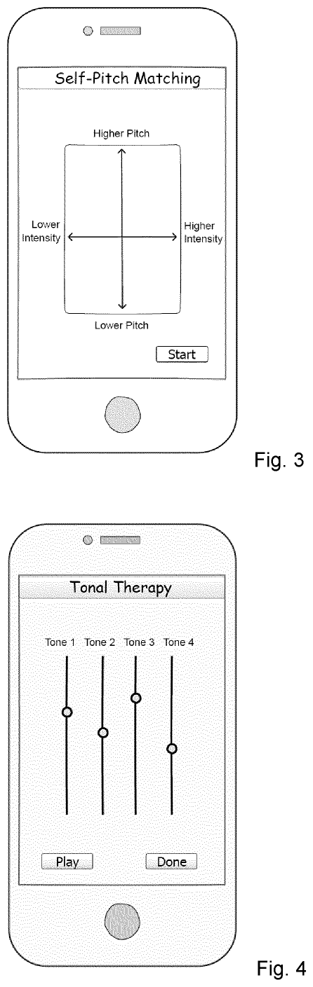

FIG. 2 shows an exemplary implementation of the tinnitus sound finder module in the patient component. The patient component is configured to guide the patient through a series of tests (here: 12), in each of which the patient component emits (preferably via the patient's headset connected to the patient component) different (here: two) tones and the patient is allowed to select between the different tones that comes closer to the patient's tinnitus.

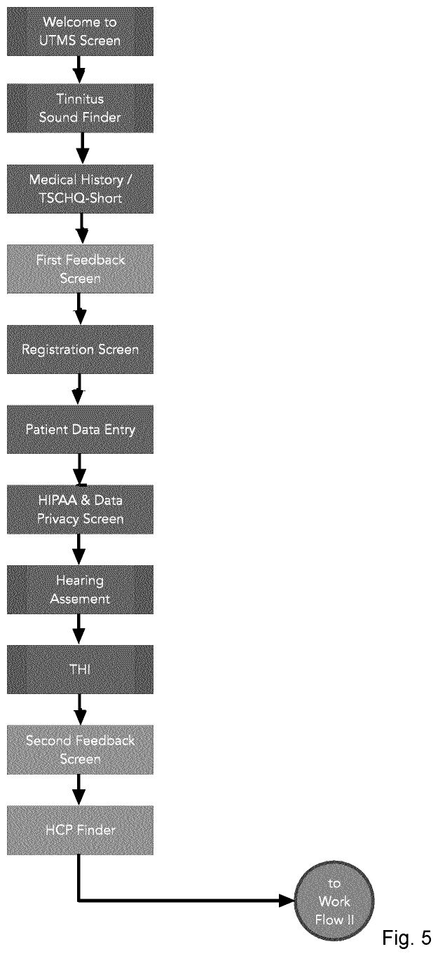

FIG. 3 shows an exemplary implementation of the self-pitch-matching module in the patient component. The patient component is configured to guide the patient through a test, in which the patient component emits (preferably via the patient's headset connected to the patient component) a tone and the patient is allowed to adapt the loudness and the pitch of the tone by gestures.

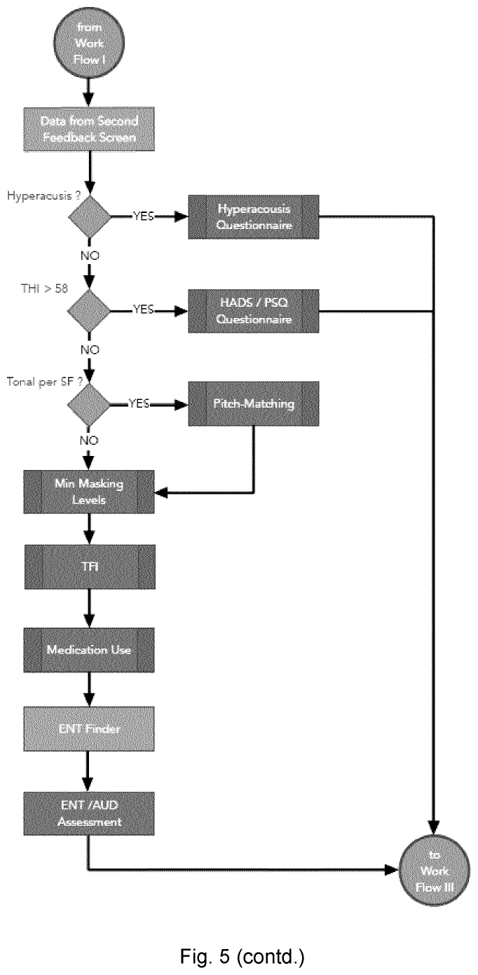

FIG. 4 shows an exemplary implementation of the tonal therapy module in the patient component. The patient component is configured to provide the following interaction items: (i) Start/stop button; (ii) Mute button; (iii) Balance adjustment button; (iv) Daily usage (timer). For tone balancing the master volume is set to 100% and the four stimulation tones are set to the tinnitus tone intensity as start intensity. Balance sliders are in middle position. Tones are played in sequential order (1,2,3,4,1,2,3,4 . . . ), active tone is marked by a differently colored slider button.

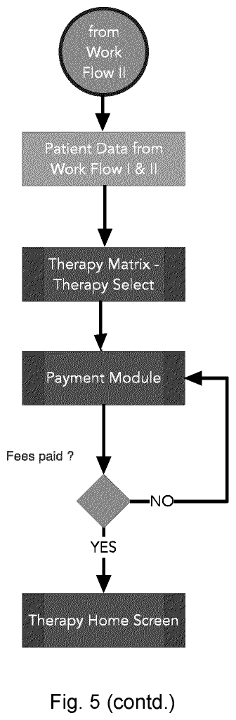

FIG. 5 shows an exemplary workflow including decision trees of the level I diagnostics, level II diagnostics and level III therapy, through which a patient may be led by the UTMS.

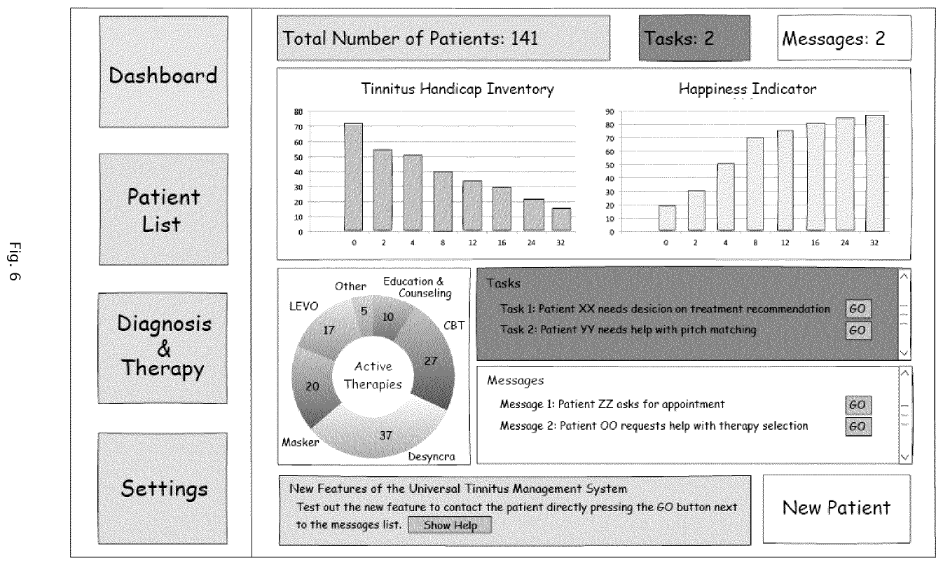

FIG. 6 shows an exemplary implementation of a dashboard as part of the HCP component. The dashboard may comprise a patient list functionality providing access to the individual patients' key information. For each patient the dashboard may allow the HCP to follow the therapy path and see improvements and problems, to add notes, to define individual therapy recommendations assisted by UMTS recommendations and/or to summarize all events in a sequential order. In the active therapy field, detailed information on the progress of the currently used therapy is listed. In the diagnosis & therapy offerings field, all available diagnostic tools and all available therapy may be listed and explained. The HCP may access information on the tool and therapy, training material to explain the tool and therapy to the patient, information on how this tool is integrated into the therapy pathway and how it influences the therapy recommendations and/or inclusion and exclusion criteria for the therapy offerings.

The UTMS as disclosed herein is a medical software-based tool that is primarily for the diagnosis and treatment of subjective tinnitus for home use. It comprises a patient component that may be composed of an app run on a mobile phone. Hence, the patient component is also referred to herein as “mobile app”. The patient component can be used by a patient who is supported by an HCP through an HCP component. The HCP component may be composed of an app run on a personal computer. Hence, the HCP component is also referred to herein as “web app”. The two components may interact once a link is established. Data between the patient component and the HCP component are exchanged in order for the HCP to review and/or define actions (in particular therapy modules to be completed) for the patient and monitor the patients therapeutic progress. The UTMS combines advanced tinnitus assessment (diagnostics), and established sound and psychological therapy, as described herein. The UTMS provides relief from the disturbance of subjective tinnitus while using the system.

The mobile app is configured to guide the patient through a number of diagnostic modules where the mobile app records data via patient data entry and results from tests performed by the patient such as answers to questionnaires or results on audiological tests. These data and results are commonly referred to as patient-specific data. The mobile app may further request from the patient to input personal information and give informed consent/HIPAA release. All data collected may be made available via online (real-time or via frequent updates) in databases, where the HCP, who may be assigned via the HCP finder module that may be included in the patient component, can see all data collected from the “assigned” patient. Measures to guarantee protection of patient's data may be implemented and may follow regulatory requirements.

The web app may then be used by the HCP to define and “prescribe and/or assign” the best therapeutic intervention for the patient based on an analysis of the data, which the UTMS system may process and display on the web app for the use by the HCP.

The UTMS is configured to be used by patients (preferably adult tinnitus patients over 18 years), one or more HCPs (for supporting and guiding the patients) and, optionally, one or more ENTs. Optionally, the UTMS may be configured to be used by one or more system administrators (for administration of the system and keeping it operating) and one or more data analysts (for evaluating the collected data for improvement of the system).

For this purpose, the UTMS comprises or consists of the following components:
a) Patient components, e.g. mobile phones on which a mobile app runs. The mobile app may be downloadable via the Apple App Store and Google Play Store (free with a subscription-based payment model). The patient component may be connectable to the patients' headsets to allow receipt of audiological signals transmitted from the UTMS.
b) One or more HCP components, e.g. personal computers on which a web app is running.
c) One or more servers, e.g. Azure.

These components form a communication network, an exemplary of which is shown in FIG. 1, whereby the right part from the UTMS server are optional components.

The UTMS may feature up to 11 diagnostic modules selected from the group consisting of Tinnitus Handicap Inventory (THI), Tinnitus Functional Index (TFI), Hospital Anxiety and Depression Scale (HADS), Hearing Assessment, Hearing Questionnaire, Pitch Matching and Pitch Quality, Hyperacusis Questionnaire, ENT Assessment, Tonality Sound Finder, Medication Use Questionnaire and Minimum Masking Levels modules. The UTMS may comprise at least 1, 2, 3, 4, 5, 6, 7, 8, 9, 10 or all of the aforementioned diagnostic modules.

The UTMS may further feature up to 6 therapy modules selected from the group consisting of Referral to an HCP for a hearing aid evaluation (Lead Generator), Patient Education Counselling, Cognitive Behavior Therapy (CBT), Tonal Sound Therapy, Masking (MML) and Noiser modules. The UTMS may comprise at least 1, 2, 3, 4, 5 or all of the aforementioned therapeutic modules.

In order to prescribe the patient the most suitable therapy, one or more of the above (self) diagnostic modules have to be completed by the user. The total time needed for completion should not exceed a certain duration.

An exemplary level I diagnostic module is the tinnitus sound finder module, which determines the nature of the tinnitus and identifies if it is a tonal tinnitus (including whistle, hum, or screech) or an atonal tinnitus (including white noise, crickets, or static). An exemplary way of implementing this module in the patient component is shown in FIG. 2.

The results of each diagnostic module are evaluated by the server and certain actions are taken automatically, while some are taken after the HCP has reviewed them. The HCP may also ask the patient to repeat a step. The following measures may then be taken: (i) If THI Score is equal to and greater than 58 the following Level II diagnostics are suggested: TFI, Medication Use Questionnaire, Hyperacusis Questionnaire, HADS & PSQ Questionnaire. (ii) If the hearing is impaired, the patient is suggested to visit the HCP and might get a hearing aid. (iii) If the tinnitus is more of a tonal nature, the Self-Pitch-Matching module is suggested as the following Level II diagnostics.

At some point in the process, the patient component may be configured to prompt the patient to select an HCP to be responsible for the respective patient and/or an ENT who may prescribe the proposed therapy. For this purpose, the patient component may include an HCP finder module and/or an ENT finder module. Optionally, the selected HCP may list his/her ENT colleagues in his/her vicinity. Level II diagnostic modules may be recommended by the server and selected by the HCP depending on the results of level I diagnostics.

An exemplary level II diagnostic module is the self-pitch-matching module, wherein the patient matches the pitch of his tinnitus sound via the patient module. An exemplary implementation of this module in the patient component is shown in FIG. 3.

After the results of the level II diagnostics are evaluated by the server, the UTMS may propose possible therapy modules and combinations, while the HCP may finally approve and/or select some or all of the proposed modules. Possible outcomes include that (i) the patient is eligible for UTMS and may advance to level III therapy; (ii) the patient is not eligible, the UTMS/HCP may reject the patient; and (iii) the patient may benefit from a hearing aid and should visit a hearing aid shop.

Once the HCP approves that the patient is suitable for treatment by UTMS (level III therapy) he may be sent an ENT form via e-mail. Alternatively, an ENT associated with an HCP may be selected from the ENT finder module, which may only be possible to run after the HCP finder module was completed and accepted by the HCP. The ENT may be granted access to the particular patient's data via the ENT module and can then complete the ENT form online and/or schedule a (real-world) appointment with the patient. In absence of a selected ENT, the patient may also print out the ENT form and gets it completed and signed by an ENT of his choice, which thereby prescribes the level III therapy. The signed form may be photographed by the app and sent to the HCP or alternatively scanned and sent via e-mail or by land mail. After the ENT form is signed the HCP assigns the proposed/prescribed therapy modules. The patient component may be configured to prompt the patient to purchase the therapy, e.g. via In-App Purchase.

The UTMS may be configured to disable that all therapy modules can be assigned simultaneously. For instance, the following therapy combinations may be allowed: Patient Education/Counselling+Masker; Patient Education/Counselling+Noiser; Patient Education/Counselling+Tonal; CBT+Noiser; CBT+Masker; or CBT+Tonal. Also, each therapy may be allowed on its own.

A level III therapy module is the tonal therapy module, an exemplary implementation of which is shown in FIG. 4.

The UTMS may be configured to lead a patient along a predetermined track through the diagnostic and/or therapy module(s). An exemplary workflow of the level I diagnostics, level II diagnostics and level III therapy is depicted in FIG. 5.

The HCP component may comprise a dashboard. The dashboard may show various information including the HCP's patients' messages, patient data, additional diagnostic assessments, etc. The dashboard may allow the HCP to review data and respond to patient requests via chat, order diagnostic modules to be repeated, assign therapy modules, and/or receive or generate reports. An exemplary implementation of the dashboard is depicted in FIG. 6.

A central function in the UTMS is a therapy matrix for supporting a decision for the most applicable therapy modules/pathway for an individual patient based on the patient data acquired through the diagnostic module(s) (e.g. questionnaires, hearing test, pitch matching, ENT assessment etc.). The therapy matrix implements established guidelines (e.g. Tunkel et al. 2014), allows identification of contraindications, allows to create a therapy proposal and supports an HCP in final therapy recommendation.

An exemplary therapy matrix is depicted in Table 1.

TABLE 1
An exemplary therapy matrix. Available therapies (top row) are linked to the available patient data (left column). If information
(e.g. a specific score range of a “test”) is not relevant (following guidelines) “NR” or indicated “I”
is marked and value 1 is given. If therapy is contraindicated because of a specific information, “C” is marked and
value 0 is given. Later-on the therapies marked with “I” will receive a “weighting factor” to assist in therapy prioritization. Ind. = indication

| | | | | | Education | | | | Tonal | | | | | |
|---|---|---|---|---|---|---|---|---|---|---|---|---|---|---|
| | | | | | & | | | | (CR) | | | | | |
| I/NR = Indicate (1) / Not Relevant ( ) | | | Hearing Aid | | Counseling | | CBT | | Therapy | | Masker | | Noiser | |
| C = Contraindicated (0) | | | Ind. | | Ind. | | Ind. | | Ind. | | Ind. | | Ind. | |
| THI | | | | | | | | | | | | | | |
| | 0 ≤ THI ≤ 36 | slight or mild | NR | | I | 1 | I | 1 | I | 1 | I | 1 | I | 1 |
| | 38 ≤ THI ≤ 56 | moderate | NR | | I | 1 | I | 1 | I | 1 | I | 1 | I | 1 |
| | 58 ≤ THI ≤ 76 | servere | NR | | I | 1 | I | 1 | I | 1 | I | 1 | I | 1 |
| | 78 ≤ THI ≤ 100 | catastrophic | NR | | I | 1 | I | 1 | C | 0 | C | 0 | C | 0 |
| TFI | | | | | | | | | | | | | | |
| | 0 ≤ TFI ≤ 31 | no or mild problem | NR | | I | 1 | I | 1 | I | 1 | I | 1 | I | 1 |
| | 32 ≤ TFI ≤ 53 | moderate problem | NR | | I | 1 | I | 1 | I | 1 | I | 1 | I | 1 |
| | 54 ≤ TFI ≤ 72 | big problem | NR | | I | 1 | I | 1 | I | 1 | I | 1 | I | 1 |
| | 73 ≤ TFI ≤ 100 | very big problem | NR | | I | 1 | I | 1 | C | 0 | C | 0 | C | 0 |
| Tonality | | | | | | | | | | | | | | |
| | tonal | | NR | | NR | | NR | | I | 1 | I | 1 | I | 1 |
| | atonal | | NR | | NR | | NR | | C | 0 | I | 1 | I | 1 |
| HADS (only if THI > 58) | | | | | | | | | | | | | | |
| | 0 ≤ HADS ≤ 7 | normal | NR | | NR | | NR | | NR | | NR | | NR | |
| | 8 ≤ HADS ≤ 10 | borderline normal | NR | | NR | | I | 1 | NR | | NR | | NR | |
| | 11 ≤ HADS ≤ 21 | abnormal | NR | | NR | | I | 1 | C | 0 | C | 0 | C | 0 |
| Hearing Loss | | | | | | | | | | | | | | |
| | YES | from questionnaire | I | 1 | I | 1 | I | 1 | NR | | NR | | NR | |
| | 0 ≤ avr. HL < 30 | normal or | NR | | NR | | NR | | NR | | NR | | NR | |
| | | moderate | | | | | | | | | | | | |
| | 30 ≤ avr. HL < 50 | severe | I | 1 | I | 1 | I | 1 | NR | | NR | | NR | |
| | 50 ≤ avr. HL | very severe | I | 1 | I | 1 | I | 1 | C | 0 | C | 0 | C | 0 |
| Pitch-Matching for CR | | | | | | | | | | | | | | |
| | not | | NR | | NR | | NR | | C | 0 | NR | | NR | |
| | available | | | | | | | | | | | | | |
| | Quality < 80 | | NR | | NR | | NR | | C | 0 | NR | | NR | |
| | 0 < PM ≤ 10000 | | NR | | NR | | NR | | I | 1 | I | 1 | I | 1 |
| | 10000 < PM | | NR | | NR | | NR | | C | 0 | I | 1 | I | 1 |
| ENT/AUD (subj./obj.) | | | | | | | | | | | | | | |
| acute Tinnitus | YES | | NR | | I | 1 | C | 0 | C | 0 | C | 0 | C | 0 |
| subjective Tinnitus | YES | | NR | | I | 1 | I | 1 | I | 1 | I | 1 | I | 1 |
| objective Tinnitus | YES | | NR | | C | 0 | C | 0 | C | 0 | C | 0 | C | 0 |
| Hyperacusis Quest. Score | | | | | | | | | | | | | | |
| | 0 < HQ < 28 | | NR | | NR | | NR | | NR | | NR | | NR | |
| | 28 ≤ HQ | | C | 0 | I | 1 | I | 1 | C | 0 | I | 1 | C | 0 |

General Min Requirements for any Therapy
Tinnitus History persistent Tinnitus checked in Patient Data Entry for all patients
Ability to read must be able to read given for all that download Apps

Based on the therapy matrix the UTMS can calculate therapy scores from the entries derived from the patient data. An exemplary way of calculating therapy scores based on the exemplary therapy matrix depicted in Table 1 above is illustrated in Table 2.

TABLE 2
Exemplary calculation of therapy scores. According to this example, all entries are multiplied.
A score of 1 may indicate a recommendation. A score of 0 may indicate a contraindication.

| | | SCORE from | HEARING | EDUCTION & | | TONAL (CR) | | |
|---|---|---|---|---|---|---|---|---|
| | | Results | AID | COUNSLING | CBT | THERAPY | MASKER | NOISER |
| THI | no or mild problem | 1 | | 1 | 1 | 1 | 1 | 1 |
| | moderate problem | 0 | | | | | | |
| | big problem | 0 | | | | | | |
| | very big problem | 0 | | | | | | |
| TFI | no or mild problem | 1 | | 1 | 1 | 1 | 1 | 1 |
| | moderate problem | 0 | | | | | | |
| | big problem | 0 | | | | | | |
| | very big problem | 0 | | | | | | |
| Tonality | tonal | 0 | | | | | | |
| | atonal | 1 | | | | 0 | 1 | 1 |
| HADS | normal | 1 | | | | | | |
| | borderline normal | 0 | | | | | | |
| | abnormal | 0 | | | | | | |
| Hearing Loss | from questionnaire | 1 | 1 | 1 | 1 | | | |
| | normal or moderate | 0 | | | | | | |
| | severe | 1 | 1 | 1 | 1 | | | |
| | very severe | 0 | | | | | | |
| Pitch Matching | PM available | 1 | | | | | | |
| | Quality too low | 1 | | 0 | 0 | 0 | 0 | 0 |
| | below 10 kHz | 0 | | | | | | |
| | above 10 kHz | 0 | | | | | | |
| ENT/ | acute | 1 | | 1 | 0 | 0 | 0 | 0 |
| audiologist | subjective | 1 | | 1 | 1 | 1 | 1 | 1 |
| | objective | 0 | | | | | | |
| Hyperacusis | yes | 0 | | | | | | |
| | no | 1 | 0 | 1 | 1 | 0 | 1 | 0 |
| | | RESULT | 0 | 0 | 0 | 0 | 0 | 0 |

Tables 3 to 5 show exemplary patient data for three different patients P2, P3 and P4.

As can be deduced from Tables 3 to 5, P2 may receive HA and Education Counselling. As P2 is “Acute”, all other therapies are contraindicated. P3 has no contraindications, i.e. is “eligible for all therapies. P2 may also see a HCP for Hearing Aid Evaluation. P4 may receive Hearing Aid. P4 is atonal, i.e. no tonal sound therapy. P4 has objective tinnitus, i.e. can be medically treated.

Table 6 provides a summary for patients P2 to P4 depicted in Tables 3 to 5 and for two additional patients, P6 and P7.

TABLE 6
Summary of calculations for model patient P2 to P7.

| | | P2 | P3 | P4 | P6 | P7 |
|---|---|---|---|---|---|---|
| Sample Patients | | | | | | |
| THI | | 25 | 45 | 50 | 60 | 85 |
| TFI | | 25 | 45 | 50 | 70 | 85 |
| Tonality | | atonal | tonal | atonal | atonal | tonal |
| HADS | | 6 | 3 | 6 | 12 | 10 |
| HEARING | | yes | no | yes | yes | no |
| Qestionnaire | | | | | | |
| HEARING | | 35 | 10 | 60 | 45 | 5 |
| Measurement | | | | | | |
| Pitch Available | | no | yes | no | no | yes |
| Pitch Matching Quality | | 0 | 90 | 0 | 0 | 90 |
| Pitch Frequency | | 0 | 4500 | 0 | 0 | 11000 |
| Acute Tinnitus | | yes | no | no | no | no |
| Subjective Tinnitus | | sub- | sub- | sub- | sub- | sub- |
| | | jective | jective | jective | jective | jective |
| HQ | | 3 | 14 | 12 | 27 | 40 |
| Therapy | | | | | | |
| Hearing Aid | HA | 1 | 1 | 1 | 1 | 0 |
| Education & | ED/C | 1 | 1 | 1 | 1 | 1 |
| Counseling | | | | | | |
| CBT | CBT | 0 | 1 | 1 | 1 | 1 |
| Tonal (CR) | TT | 0 | 1 | 0 | 0 | 0 |
| Therapy | | | | | | |
| Masker | MSK | 0 | 1 | 1 | 0 | 0 |
| Noiser | NOI | 0 | 1 | 1 | 0 | 0 |

| | | | | | |
|---|---|---|---|---|---|
| Offered to HCP | P2 | P3 | P4 | P6 | P7 |

In Table 6, the five patients P2 to P7 are listed in the five columns. For each patient the scores of the questionnaires (e.g. THI) and the corresponding results of the diagnosis modules are given in the first part of the table. For each therapy module (here 6 therapy modules) the score (either 1 or 0) is calculated using the therapy matrix. “1” corresponds to a recommendation, “0” corresponds to no recommendation, namely a contra-indication.

This way the UTMS is capable to provide a therapy proposal. The therapy proposal may then be transmitted to and displayed on the HCP component. The therapy proposal may include therapy modules for which the respective patient is eligible, and therapy modules, which are contraindicated with the respective patient. Using the HCP component, the HCP can select one or more among the eligible therapy modules. The contraindicated therapy modules may be displayed in a form in which they cannot be selected by the HCP. For instance, they may be grayed-out.

The therapy modules may be divided into groups. For instance, the therapy modules may be divided in three groups. A first group may include the hearing aid module. A second group may include the education and counselling module and the CBT module. A third group may include the tonal therapy module, the masker module and the noiser module. The UTMS may be configured to allow the HCP to select only one among therapy module of each group. In this case, once the HCP has selected one module in a group, the other module(s) in the same group may be greyed-out. The reasoning behind such grouping is that only one sound-based module can be done at one time. Also, the CBT module bases on the patient education & counselling module, therefore a predefined order (e.g. first education & counselling module, second CBT module) is given by this measure.

1. A universal tinnitus management system (UTMS) for diagnosis and/or treatment of subjective tinnitus, or for support of a health care professional (HCP) in the diagnosis and/or treatment of subjective tinnitus, the UTMS comprising:
a) an HCP component,
b) a plurality of patient components, and
c) optionally an ear, nose and throat doctor (ENT) component,
wherein the UTMS is configured to facilitate communication between the HCP component, the plurality of patient components and, if present, the ENT component, via a server,
wherein the HCP component is configured to facilitate management of the plurality of patient components,
wherein each of the plurality of patient components is configured to be assigned to a specific patient and to authenticate the respective patient component to the UTMS, wherein the respective patient component is configured to request patient-specific data from the respective patient,
wherein the UTMS is configured to process the patient-specific data and communicate them to the HCP component and, if present, the ENT component, wherein the UTMS is configured to draw up a diagnosis and/or a therapy proposal based on a predetermined therapy matrix using the patient-specific data as an input, and
wherein the patient component is configured to display the diagnosis and/or the therapy proposal.

2. The UTMS of claim 1, wherein the UTMS is configured to communicate the diagnosis and/or the therapy proposal to the HCP component and/or, if present, the ENT component for confirmation, rejection or revision of the diagnosis and/or the therapy by a HCP and/or an ENT before the diagnosis and/or the therapy proposal is displayed on the patient component.

3. The UTMS of claim 1, wherein the UTMS comprises at least one diagnostic module for determining, conducting and/or receiving results from of at least one of medical history, Tinnitus Handicap Inventory (THI), hearing assessment, tonality sound finder, medication use questionnaire, Tinnitus Functional Index (TFI), self-pitch matching, hyperacusis questionnaire, Hospital Anxiety and Depression Scale (HADS), perceived stress questionnaire (PSQ), ENT assessment, hearing questionnaire, pitch quality, and minimum masking levels, and wherein the patient-specific data include the respective patient's results from the completion of the at least one diagnostic module.

4. The UTMS of claim 3, wherein the patient component is configured to invite the respective patient to perform at least one of the THI, the hearing assessment, the hearing questionnaire and the tonality sound finder modules in a first step.

5. The UTMS of claim 4, wherein, if in the first step the respective patient is determined to have hyperacousis, a THI index equal or above a predefined threshold or a tonal Tinnitus, the patient component is configured to invite the respective patient to perform at least one of the hyperacousis questionnaire, the HADS and the PSQ questionnaire and the pitch matching modules in a second step.

6. The UTMS of claim 4 or 5, wherein, if in the first step the respective patient is determined to have no hyperacousis, a THI index below a predefined threshold, and no tonal Tinnitus, the patient component is configured to invite the respective patient to perform at least one of the minimum masking levels, the TFI and the medication use questionnaire modules in a third step.

7. The UTMS of claim 6, wherein the third step includes an invitation of the respective patient by the patient module to perform the ENT assessment module.

8. The UTMS of claim 4, wherein the UTMS is configured to draw up the diagnosis and/or the therapy proposal based on the results of the diagnostic modules and/or therapeutic modules as the patient-specific data and using the predetermined therapy matrix.

9. The UTMS of claim 1, wherein the UTMS is configured to determine whether a therapy is contraindicated based on the predetermined therapy matrix and/or to determine weighting factors for different therapy options for assisting in therapy priorization based on the predetermined therapy matrix, and communicate the respective results to the HCP component.

10. The UTMS of claim 1, wherein the UTMS comprises at least one therapy module for proposing to the respective patient component at least one of referral to an HCP for a hearing aid evaluation, patient education counselling, cognitive behavior therapy (CBT), tonal sound therapy, masking (MML) and noiser usage.

11. The UTMS of claim 1, wherein the therapy proposal includes more than one therapy modules to be performed by the respective patient, and wherein the patient component is configured to provide an overview of the therapy modules to be performed.

12. The UTMS of claim 1, wherein the patient component is configured to request patient-specific data at different time points, and wherein the UTMS is configured to track over time patient compliance with the therapy proposal and/or therapy progress.

13. (canceled)

14. A method of diagnosing and/or providing a therapy proposal for treating a subject having subjective tinnitus by means of universal tinnitus management system (UTMS) as defined by claim 1, comprising the steps:
registration of the subject as a new patient of the UTMS,
providing patient-specific data of the subject to the UTMS, and
drawing up a diagnosis and/or a therapy proposal based on a predetermined therapy matrix using the patient-specific data as an input.

15. The method of claim 14, further comprising:
tracking over time the subject's compliance with the therapy proposal and/or therapy progress using the HCP component.
